(19) United States
(12) Patent Application Publication
(10) Pub. No.: US 2015/0182203 A1
(43) Pub. Date: Jul. 2, 2015

(54) SWAB SHAFT AND METHOD OF WRAPPING SWAB MATERIAL ONTO IT

(71) Applicant: MML Diagnostics Packaging, Inc., Troutdale, OR (US)

(72) Inventor: Dale Pestes, Troutdale, OR (US)

(21) Appl. No.: 14/146,261

(22) Filed: Jan. 2, 2014

(51) Int. Cl. A61B 10/02; B32B 37/12; B32B 37/30; B32B 37/14

(52) U.S. Cl. CPC A61B 10/02; B32B 37/142; B32B 37/1292; B32B 37/30; B32B 2535/00

(57) ABSTRACT

A shaft for a cell collection swab has a plurality of anchors protruding from its tip which facilitate attaching swab material to the shaft.

### BACKGROUND

In order to diagnose certain urinary tract or genital diseases or to perform certain types of DNA testing, it is necessary to collect cell samples from the patient's urethra. Swabs for doing this are made by wrapping swab material around the tip of a shaft by bringing the swab material into contact with the shaft while it is being rotated. Swabs for this purpose are described in detail in U.S. Pat. No. 5,623,942, which is owned by applicant and is incorporated herein by reference. One difficulty with making these cell collection swabs of this type is that the swab material does not always get tightly wrapped around the shaft resulting in a defective swab.

### BRIEF SUMMARY

The subject invention relates to a shaft for a cell collection swab having an elongate probe portion which has a plurality of anchors protruding from it which are configured to capture the swab material when it is placed over a rotating shaft.

### BRIEF DESCRIPTION OF THE DRAWINGS

FIG. 1 is a swab utilizing the shaft of the subject invention.

FIG. 2 is a cross-sectional view taken on the line 2-2 of FIG. 1.

FIG. 3 is a cross-sectional view taken on the line 3-3 of FIG. 1.

FIG. 4 is a detailed view of a swab shaft showing the anchors which are an element of the invention.

FIG. 5 is a foreshortened view of the swab shaft showing the anchors.

FIGS. 6-10 show the method by which swab material is attached to the swab shaft.

### DETAILED DESCRIPTION OF PREFERRED EMBODIMENTS

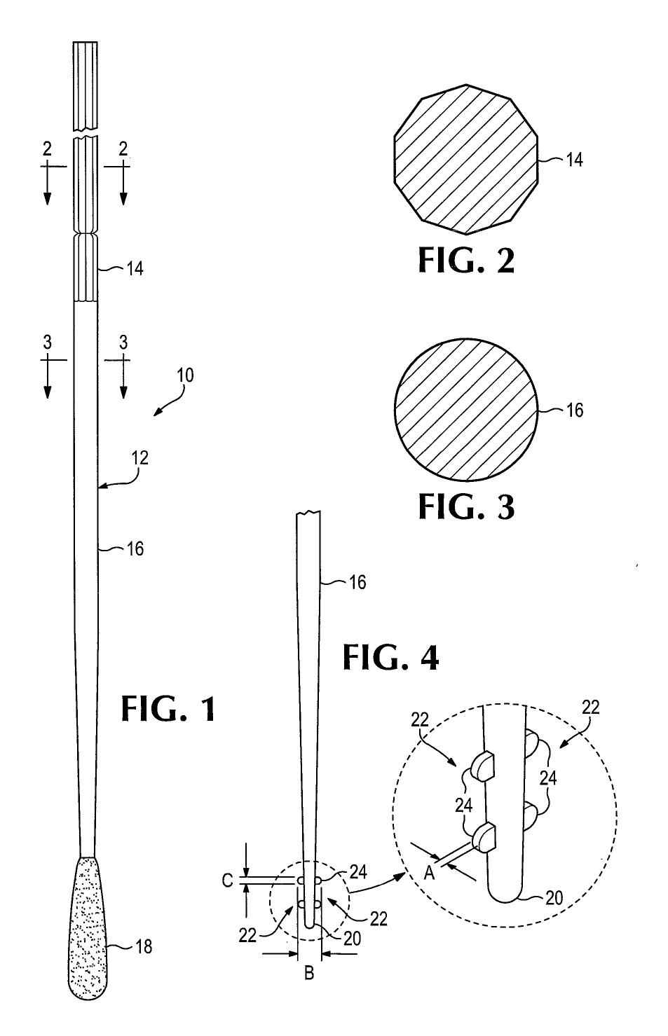

Referring now to FIG. 1 of the drawings, a swab 10 for collecting cell samples from a male urethra comprise a unitary elongate shaft 12. The shaft is divided into a handle portion 14 and a probe portion 16. Swab material 18 is attached to the tip of the probe portion. This basic swab is disclosed in U.S. Pat. No. 5,623,942. In the embodiment illustrated the handle portion is polygonal in a cross-section but it could be circular as well.

Referring now to FIG. 4, a plurality of protruding anchors 22 protrude from the shaft tip 20. In the embodiment illustrated there are two sets of anchors which are oriented approximately 180 degrees from one another. Each set includes two longitudinally aligned anchors.

In the embodiment illustrated, the anchors have spaced apart planar sides 24 which are separated from one another by a distance A. Distance A is between 0.005 inches and 0.015 inches and preferably is around 0.010 inches. The anchors have rounded ends. The anchors protrude from the tip by an amount such that the distance B between the ends of opposed anchors is approximately equal to the diameter of the handle portion 14. The anchors 22 have a width C of approximately 0.025 inches. The center line of the first anchor in each set is located approximately 0.010 from the end of the tip and the center line of the second anchor in each set is located approximately 0.020 from the end of the tip. The size, shape and location of the anchors can vary from the embodiment shown in the drawings and described above.

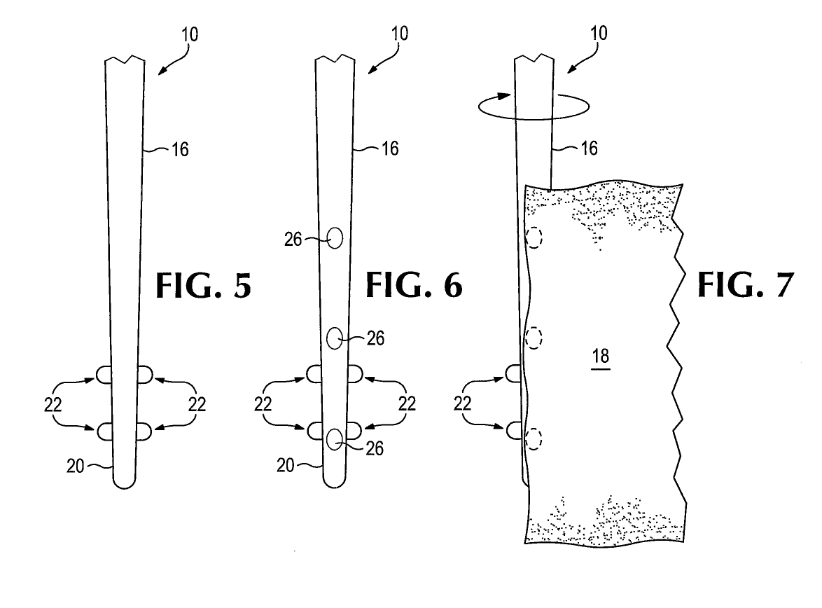

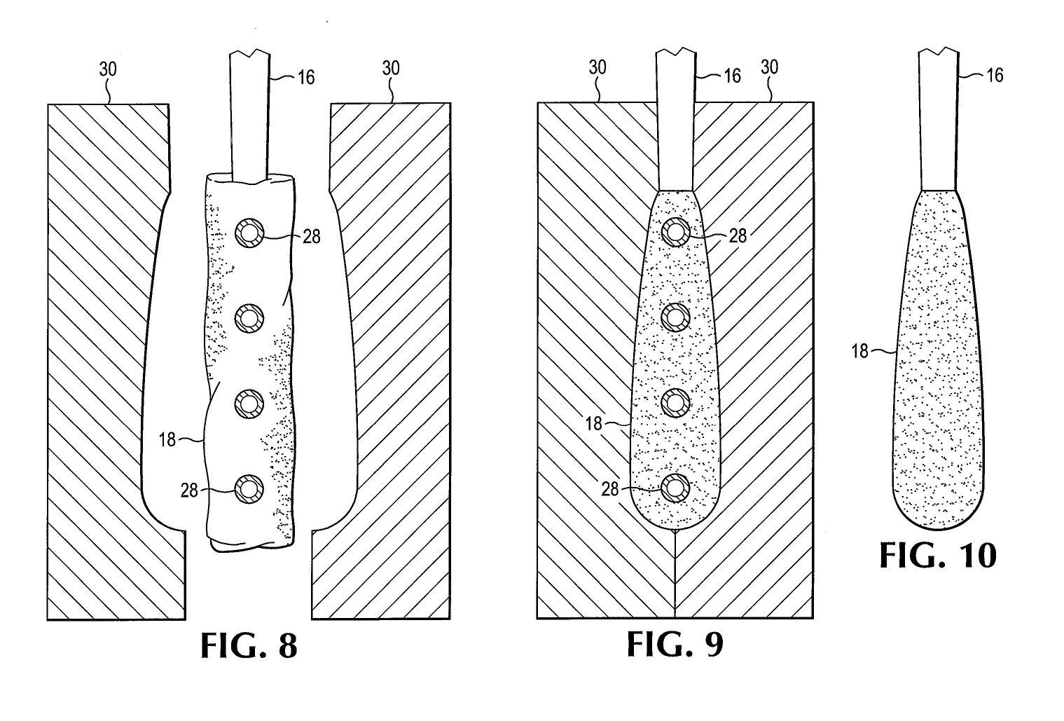

Referring now to FIGS. 5-10, before swab material 18 is attached to the shaft 12, several spots of liquid adhesive 26 are deposited on the portion of the tip 20 that the swab material will cover, FIG. 6. The piece of swab material 18 then is placed on the tip on top of the adhesive spots 26 while the shaft is rotating, FIG. 7. While the adhesive will generally cause the swab material to adhere to the tip 20, rotate with the shaft, and become tightly wrapped around it, the anchors 22 more positively engage the swab material and hold it on the rotating shaft. After the swab material 18 is fully wrapped on to the tip 20 a water, starch mixture is sprayed onto the swab material from a series of nozzles 28 and a die 30 is closed over the swab material to form it to a desired oblong shape, FIG. 9. After the water starch mixture has dried the completed swab is removed from the die 30.

What is claimed is:

1. A shaft for a cell collections swab comprising:
(a) An elongate handle portion;
(b) An elongate probe portion having a first end which is connected to said handle portion and a second end which is configured to have swab material attached to it; and
(c) A plurality of anchors extending radially outwardly from said second end.

2. The shaft of claim 1 wherein said elongate probe portion is circular in cross-section and there are two sets of said anchors with one set being offset approximately 180 degrees from the other set.

3. The shaft of claim 2 wherein said probe portion has a distal extremity and is tapered to a smaller diameter towards the distal extremity thereof.

4. The swab of claim 1 wherein said anchors have spaced apart planar sides.

5. The shaft of claim 4 wherein said anchors have rounded ends.

6. The shaft of claim 4 wherein the distance between said spaced apart sides is between 0.005 inches and 0.015 inches.

7. The shaft of claim 6 wherein the distance between said spaced apart sides is 0.010 inches.

8. A method of wrapping swab material onto a probe shaft comprising:
(a) providing an elongate shaft having a plurality of protruding anchors located proximate the tip thereof;
(b) rotating said shaft about its elongate axis;
(c) placing a small amount of adhesive on said shaft adjacent said anchors;
(d) placing a sheet of swab material against the rotating shaft over said tip;
(e) allowing said sheet of swab material to be captured by said anchors and wrapped around said tip;
(f) after said swab material is completely wrapped onto said shaft, spraying said swab material with a starch water solution;
(g) then compressing said swap material to provide a contoured swab tip.
